(19) United States
(12) Patent Application Publication
(10) Pub. No.: US 2021/0015738 A1
(43) Pub. Date: Jan. 21, 2021

(54) ORAL DISSOLVABLE FILM CONTAINING PSYCHEDELIC COMPOUND

(71) Applicant: Concept Matrix Solutions, Woodland Hills, CA (US)

(72) Inventors: Tony LaRosa, Woodland Hills, CA (US); Robert Davidson, Woodland Hills, CA (US); David Reid, Woodland Hills, CA (US)

(21) Appl. No.: 16/947,005

(22) Filed: Jul. 14, 2020

Related U.S. Application Data
(60) Provisional application No. 62/875,075, filed on Jul. 17, 2019.

(51) Int. Cl. A61K 9/00; A61K 31/675; A61K 31/4045

(52) U.S. Cl. CPC A61K 9/006; A61K 31/4045; A61K 31/675

(57) ABSTRACT

Provided is an oral dissolvable film that includes a psychedelic compound. Also provided is a method of treating in a subject a disease or disorder ameliorated by a psychedelic compound, that includes orally administering to a subject an oral dissolvable film that includes a therapeutically effective amount of the psychedelic compound. Also provided is a method of orally administering to a subject an oral dissolvable film that includes a therapeutically effective amount of the psychedelic compound. Also provided is a method of orally administering to a subject an oral dissolvable film that includes a low dose (e.g., microdose or sub-therapeutic dose) of the psychedelic compound.

### RELATED U.S. APPLICATION DATA

This application claims priority to provisional patent application No. 62/875,075 filed on Jul. 17, 2019, the contents of which are incorporated by reference herein in its entirety.

### SUMMARY

The present invention provides for an oral dissolvable film that includes: (i) a flowable water-soluble or water swellable film-forming matrix that includes a polymer, and (ii) psychedelic compound selected from the group consisting of psilocybin, psilocin, mescaline, lysergic acid diethylamide (LSD), ketamine, salvinorin A, ibotenic acid, muscimol, N,N-dimethyltryptamine (DMT), 3,4-methylenedioxymethamphetamine (MDMA), methyl diethanolamine, also known as N-methyl diethanolamine (MDEA), 3,4-methylenedioxy amphetamine (MDA), and combinations thereof.

The present invention also provides for a method of treating in a subject a disease or disorder ameliorated by a psychedelic compound. The method includes orally administering to the subject an oral dissolvable film described herein, in an amount and for a period of time sufficient to effectively treat the disease or disorder.

The present invention also provides for a method of treating in a subject a psychological or neurological disorder. The method includes orally administering to the subject an oral dissolvable film described herein, in an amount and for a period of time sufficient to effectively treat the psychological or neurological disorder.

The present invention also provides for a method of treating in a subject at least one of obsessive compulsive disorder (OCD), depression, pain, irritability, fibromyalgia, post-traumatic stress disorder (PTSD), cluster headaches, paranoia, psychosis, anxiety, panic attacks, flashbacks, smoking addiction, alcohol addiction, drug addiction, and cocaine addiction. The method includes orally administering to the subject an oral dissolvable film described herein, in an amount and for a period of time sufficient to effectively treat the disease or disorder.

The present invention also provides for a method of improving creativity, boosting physical energy level, attaining emotional balance, improving the mood, and/or increasing performance on problem-solving tasks. The method includes orally administering to the subject an oral dissolvable film described herein, in an amount and for a period of time sufficient to effectively improve creativity, boost physical energy level, attain emotional balance, improve the mood, and/or increase performance on problem-solving tasks.

The present invention also provides for a method of orally administering to a subject an oral dissolvable film described herein, wherein the oral dissolvable film includes a low dose or microdose of the psychedelic compound.

The present invention also provides for a method of administering to a subject a low dose or microdose of a psychedelic compound. The method includes orally administering an oral dissolvable film described herein, wherein the oral dissolvable film includes a low dose or microdose of the psychedelic compound.

### DETAILED DESCRIPTION

The present invention is directed to an oral dissolvable film that includes a psychedelic compound. The present invention is also directed to a method of treating in a subject a disease or disorder ameliorated by a psychedelic compound, that includes orally administering to a subject an oral dissolvable film that includes a therapeutically effective amount of the psychedelic compound. The present invention is also a method of orally administering to a subject an oral dissolvable film that includes a therapeutically effective amount of the psychedelic compound. The present invention is also a method of orally administering to a subject an oral dissolvable film that includes a low dose (e.g., microdose or sub-therapeutic dose) of the psychedelic compound.

The words “comprise,” “comprising,” “include,” “including,” and “includes” when used in this specification and claims are intended to specify the presence of stated substances, features, integers, components, or steps, but they do not preclude the presence or addition of one or more other substances, features, integers, components, steps, or combinations thereof.

The term “oral dissolvable film” (and alternative terms such as “oral soluble film,” “oral dissolvable strip,” “oral soluble strip,” “oral film,” “oral strip,” etc.) refers to a dissolvable film specifically configured for oral administration. The oral dissolvable film is self-supporting, or in other words, is able to maintain its integrity and structure in the absence of a separate support. Oral dissolvable films are composed of pharmaceutically acceptable ingredients that are edible or ingestible. The oral dissolvable film can be configured for multi- or unidirectional release. Similar in size and shape to a postage stamp, oral dissolvable films are designed for oral administration, with the user placing the film on the tongue (enteric), under the tongue (sublingual), on the oral mucosa (mucosal), against the inside of the cheek (buccal), or on the gums (gingival). Aside from the enteric route, these drug delivery options allow the active ingredient to bypass the first pass metabolism, thereby making the active ingredient more bioavailable. As the film dissolves, the active ingredient can enter the blood stream enterically, mucosally, buccally, gingivally, and/or sublingually. As such, the oral dissolvable film is typically prepared using hydrophilic polymers (e.g., film forming polymers) that dissolve on the tongue or buccal cavity, delivering the active ingredient to the systemic circulation via dissolution when contact with liquid is made. Oral film drug delivery accordingly uses a dissolving film to administer active ingredients via absorption in the mouth (buccally, sublingually, or gingivally) and/or via the small intestines (enterically). Especially for active ingredients which are metabolized extensively by the first-pass effect, oral films described herein provide an opportunity for a faster-acting and better absorption profile.

The oral dissolvable film described herein can include a single film matrix. Alternatively, the oral dissolvable film can include multiple (e.g., 2, 3, 4, etc.) film matrices. When the oral dissolvable film includes multiple film matrices, any one or more of the film matrices can independently be composed of the same substances present in the other film matrices. Alternatively, any one of the matrices can independently be composed of different substances present in the other film matrices (e.g., non-uniform distribution of substances in the thickness direction among the multiple film matrices).

The oral dissolvable film described herein includes a polymeric matrix formed from a film forming agent (e.g., film-forming polymer), active pharmaceutical ingredient (API), and solvent. Optional additional excipients (alternatively referred to as “additives”) used to manufacture the oral film can include one or more of: mucoadhesive polymer, plasticizer, binder, filler, bulking agent, saliva stimulating agent, stabilizing and thickening agent, gelling agent, flavoring agent, taste masking agent, coloring agent, pigment, lubricant, release modifier, adjuvant, sweetening agent, solubilizer & emulsifier, fragrance, emulsifier, surfactant, pH adjusting agent, buffering agent, lipid, glidant, stabilizer, antioxidant, anti-tacking agent, humectant, solvent, permeation enhancer, and preservative. Suitable excipients or additives that can be used in the formulation of oral films are described in, e.g., Lachman, et al., “The Theory and Practice of Industrial Pharmacy,” 4th Edition (2013); Rowe et al., “Handbook of Pharmaceutical Excipients,” 8th Edition (2017); and Remington, “The Science and Practice of Pharmacy,” 22nd Edition (2015). From the regulatory perspectives, all excipients and additives used in the formulation of the oral films described herein should preferably be approved for use in oral pharmaceutical dosage forms.

It is appreciated that those of skill in the art understand that any substance employed in the slurry and/or oral dissolvable film can have multiple functions. However, unless the substance is otherwise indicated as having only a single function, reference to that substance as having a specified function is nonetheless appropriate and non-limiting, with the understanding that it may also have one or more additional functions. It is also appreciated that those of skill in the art understand that when feasible, the slurry and/or oral dissolvable film will preferably include substances that serve multiple desired purposes (e.g., possess multiple desired functions). In doing so, an oral dissolvable film can therefore be obtained that weighs less, dries quicker, disintegrates faster, and/or allows for a higher load of active ingredient.

The term “slurry” refers to a mixture of solids suspended and/or dissolved in liquid, and is suitable to be extruded, cast onto a substrate, and cured to form a dissolvable film. The solids and liquid will expectedly include those substances used to manufacture the oral dissolvable film. The solid substances employed in the manufacture of the oral dissolvable film can be dissolved and/or suspended in the liquid. The oral dissolvable film can be formed by curing the cast slurry, wherein the curing can be carried out at an elevated temperature for a period of time. In doing so, an appreciable amount of the solvent (e.g., water) will be removed.

The term “matrix,” “film matrix,” or “polymeric matrix” refers to the matrix of film forming polymer having the active ingredient embedded therein. In addition to the active ingredient, the polymeric matrix can further include additional substances embedded therein. These would include any one or more of those substances used to form the slurry. As the cast slurry is cured to provide a dissolvable film, a polymeric matrix is formed which contains the active ingredient (and optionally one or more additional substances) embedded therein. For example, when the slurry contains an active ingredient, film forming polymer, solvent, binder, and plasticizer, upon casting and curing to provide the dissolvable film, a polymeric matrix is formed which can contain each of the active ingredient, film forming polymer, solvent, binder, and plasticizer. Alternatively, the polymeric matrix can be formed containing each of the active ingredient, film forming polymer, binder, and plasticizer (i.e., no solvent).

The term “pharmaceutically acceptable” refers to those compounds, excipients, active ingredients, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio. This would include, e.g., those substances present on the FDA's Inactive Ingredient Database (IID) (https://www.accessdata.fda.gov/scripts/cder/iig/index.Cfm) as well as those substances considered to be generally recognized as safe (GRAS).

The term “solvent” refers to a substance that dissolves a solute, resulting in a solution. With the oral dissolvable film described herein, the solute can include, e.g., the film forming polymer, the active ingredient and excipients such as, e.g., plasticizer, sweetener, flavoring agent, binder, preservative, coloring agent, and pH adjusting agent. Additionally, with the oral dissolvable film described herein, the slurry can be a solution. As such, the solvent is employed to form the slurry by dissolving the desired substances to be included in the slurry (and subsequently the oral dissolvable film). The solvent can be an aqueous solvent, thereby including water. Alternatively, the solvent can include an organic liquid, such as ethanol. The water present in the oral dissolvable film described herein can function as a solvent. Additionally, the water can further optionally function as a plasticizer, process aid, or combination thereof. The term “solvent” also embraces “co-solvent,” which is a substance, present along with the solvent, that aids, facilitates, or promotes the dissolving of the solute, to provide the solution (e.g., slurry). The co-solvent will typically include an organic liquid, such as glycerin, propylene glycol, polyethylene glycol, or a combination thereof.

The term “plasticizer” refers to a substance that, when added to polymer(s), they make the polymer more pliable and soft, enhancing the flexibility and plasticity of the films while reducing the brittleness. The plasticizer is believed to permeate the polymer structure, disrupting intermolecular hydrogen bonding, and permanently lowers intermolecular attractions. Plasticizers can be used to allow initial film forming, to reduce the brittleness, and improve the processability and flexibility of the resulting film, thereby avoiding cracking, e.g., during the curing process. Suitable plasticizers include, e.g., glycerin, water, polyethylene glycol, honey, propylene glycol, monoacetin, triacetin, triethyl citrate, sorbitol, 1,3-butanediol, D-glucono-1,5-lactone, diethylene glycol, castor oil, and combinations thereof.

The term “sweetener” or “sweetening agent” refers to a substance that provides a sweet taste. The sweetener can be natural or artificial. Suitable sweeteners include sugars (e.g., glucose, corn syrup, fructose, and sucrose) as well as sugar substitutes (e.g., honey, honey granules, aspartame, neotame, acesulfame potassium (Ace-K), saccharin, sodium saccharine, advantame, sucralose, monk fruit extract (mogrosides), stevia, rebaudioside A, sorbitol, xylitol, and lactitol).

The term “flavoring agent” refers to a substance used to impart a flavor, e.g., to improve the attractiveness and acceptance by the subject. The basic taste sensations are salty, sweet, bitter, sour, and umami. Flavors may be chosen from natural and synthetic flavorings. An illustrative list of such agents includes volatile oils, synthetic flavor oils, flavoring aromatics, oils, liquids, oleoresins or extracts derived from plants, leaves, flowers, fruits, stems and combinations thereof. The flavoring agent can include, e.g., one or more of honey, anise, cherry, mint, peppermint, spearmint, menthol, levomenthol, watermint, gingermint, lemongrass, cardamom, sage, cinnamon, ginger, allspice, clove, eugenol, orange, wintergreen, lemon, lime, tangerine, ginger, and nutmeg. The flavoring agent can be available as a solid (e.g., powder), as a liquid (e.g., oil), or a combination thereof.

The term “taste masking agent” refers to a substance used to mask the unpleasant taste of a substance present in the formulation, to improve the attractiveness and acceptance by the subject. For example, the taste masking agent can refer to a substance used to mask the bitter taste of the active ingredient. With the oral dissolvable films described herein, the taste masking agent can include, e.g., at least one of honey, anise, mint, peppermint, cinnamon, magna sweet, citrus, and fruit (e.g., cherry). In addition to imparting a flavor, the flavoring agent can optionally also mask the taste of any unpleasant or bitter tasting substances (e.g., the active ingredient) present in the oral dissolvable film. In such embodiments, the same substance can serve as both a flavoring agent and a taste masking agent.

The term “binder” refers to a substance, typically a polymer, used to hold the ingredients together. Binders ensure that the oral dissolvable films can be formed with the requisite mechanical strength. The binders also provide the requisite volume to low amount of active present in dissolvable films. The presence of the binder also facilitates the formation of the cured film. As such, the binder includes those substances, which when present in the cast slurry and upon curing, will effectively provide for a cured film. The binder may also be referred to as a “film forming agent,” or more specifically a “film forming polymer” when it is a polymer. The polymer can be a natural polymer or a synthetic polymer. Natural polymers include, e.g., pullulan, sodium alginate (Na alginate), pectin, gelatin, chitosan, and maltodextrin. Synthetic polymers include, e.g., hydroxpropyl cellulose (HPC), hydroxpropyl methylcellulose (HPMC), carboxymethyl cellulose (CMC), sodium carboxymethylcellulose (CMC-Na), microcrystalline cellulose (MCC), polyvinyl alcohol (PVA), polyethylene oxide (PEO), polyvinylpyrrolidone (PVP), and Kollicoat® (e.g., Kollicoat® Protect or Kollicoat® IR).

The term “mucoadhesive agent” refers to a substance that, upon contact with a mucosal surface (e.g., oral cavity), will adhere therein. The mucoadhesive agent, when placed in the oral cavity in contact with the mucosa therein, will adhere to the mucosa. The mucoadhesive agent permits a close and extended contact of the composition of the oral dissolvable film with the mucosal surface of the subject, by promoting adherence of the composition to the mucosa, and facilitating the release of the active ingredient from the composition. The mucoadhesive agent can be a polymeric compound, such as a cellulose derivative but it can be also a natural gum, alginate, pectin, or such similar polymer. The concentration of the mucoadhesive agent can be adjusted to vary the length of time that the film adheres to the mucosa or to vary the adhesive forces generated between the film and mucosa. Mucoadhesive agents include, e.g., carboxymethyl cellulose (CMC), carboxymethyl cellulose sodium (CMC-Na), polyvinyl alcohol, polyvinyl pyrrolidone (povidone), sodium alginate, methyl cellulose, hydroxyl propyl cellulose, hydroxypropylmethyl cellulose, polyethylene glycols, carbopol, polycarbophil, carboxyvinyl copolymers, propylene glycol alginate, alginic acid, methyl methacrylate copolymers, tragacanth gum, guar gum, karaya gum, ethylene vinyl acetate, dimethylpolysiloxanes, polyoxyalkylene block copolymers, pectin, chitosan, carrageenan, xanthan gum, gellan gum, gum Arabic, locust bean gum, and hydroxyethylmethacrylate copolymers.

The term “preservative” refers to a substance that is added to prevent decomposition by microbial growth or by undesirable chemical changes. Some typical preservatives used in pharmaceutical formulations include: antioxidants like vitamin A, vitamin E, vitamin C, vitamin C palmitate, retinyl palmitate, and selenium; the amino acids cysteine and methionine; citric acid and sodium citrate; synthetic preservatives like the parabens: methyl paraben and propyl paraben. With the oral dissolvable films described herein, the preservative can include, e.g., any one or more of sodium benzoate, benzoic acid, sodium nitrite, sodium sorbate, potassium sorbate, and ascorbic acid.

The term “coloring agent,” “colorant,” or “pigment” refers to a substance used to impart a color, e.g., to improve the appearance and attractiveness by the subject. Color consistency can be significant, as it allows easy identification of a medication to the subject. Furthermore, colors often improve the aesthetic look and feel of medications. By increasing these organoleptic properties a subject is more likely to adhere to their schedule and therapeutic objectives will also have a better outcome for the subject.

The term “pH adjusting agent” refers to a substance that, when added to an aqueous solution (e.g., slurry), will change the pH. For example, the pH adjusting agent can be an acid, such that when added to an aqueous solution (e.g., slurry), it will decrease the pH. Alternatively, the pH adjusting agent can be a base, such that when added to an aqueous solution (e.g., slurry), it will increase the pH. The base can be an organic base (e.g., sodium bicarbonate) or inorganic base (e.g., sodium hydroxide), and the acid can be at least one of an inorganic acid (e.g., hydrochloric acid) and/or an organic acid (e.g., citric acid, malic acid, tartaric acid, etc.).

The term “buffering agent” refers to a weak acid or weak base used to maintain the pH (e.g., acidity or basicity) of a solution (e.g., slurry) near a chosen value after the addition of another acid or base. That is, the function of a buffering agent is to prevent a rapid change in pH when acids or bases are added to the solution (e.g., slurry). Buffering agents have variable properties—some are more soluble than others; some are acidic while others are basic. The acid can be an organic acid, mineral acid, or combination thereof. Likewise, the base can be an organic base, inorganic base, or combination thereof.

The terms “filler” and “bulking agent” refer to substances that add bulk to the pharmaceutical dosage form, making very small active ingredient components easy for consumer to take. Fillers are added to pharmaceutical dosage form to help with the manufacturing and stabilization of these products. Fillers bind and stabilize the dosage form. They do not alter or impact the effectiveness of the active pharmaceutical ingredient (API). Examples include: lactose, glucose, plant cellulose, microcrystalline cellulose (MCC), and calcium carbonate.

The term “saliva stimulating agent” or “salivary stimulant” refers to a substance capable of increasing the production of saliva, thereby increasing salivary flow rate. Suitable saliva stimulating agents include organic acids (e.g., ascorbic acid and malic acid), parasympathomimetic drugs (e.g., choline esters such as pilocarpine hydrochloride and cholinesterase inhibitors), physostigmine, and other substances (e.g., xylitol, xylitol/sorbitol, and nicotinamide).

The term “stabilizing and thickening agent” or “gelling agent” refers to substances employed to improve the viscosity and consistency of the slurry before casting. Active ingredient content uniformity is often a requirement for all dosage forms, particularly those containing low dose highly potent active ingredients. To uniquely meet this requirement, oral dissolvable film formulations can contain uniform dispersions of active ingredient throughout the whole manufacturing process. Examples of stabilizing and thickening agents include, e.g., alginic acid, sodium alginate, potassium alginate, ammonium alginate, calcium alginate, agar, carrageenan, locust bean gum, pectin, and gelatin.

The term “solubilizer & emulsifier” or “emulsifier” refers to a substance capable of forming or promoting an emulsion. In particular reference to the oral dissolvable films described herein, the emulsifier promotes the separation of phases (e.g., aqueous and lipids), while allowing them to be mixed. Suitable emulsifiers include, e.g., Polysorbate 80, glycerin, propylene glycol, and polyethylene glycol.

The term “emulsion” refers to a mixture of two or more liquids that are normally immiscible (nonmixable or unblendable). Emulsions are part of a more general class of two-phase systems of matter called colloids. Although the terms colloid and emulsion are sometimes used interchangeably, emulsion should be used when both the dispersed and the continuous phase are liquids. In an emulsion, one liquid (the dispersed phase) is dispersed in the other (the continuous phase). Examples of emulsions include vinaigrettes, milk, mayonnaise, and some cutting fluids for metal working. The photo-sensitive side of photographic film is an example of a colloid.

The term “lipid” refers to a group of naturally occurring molecules that include fats, waxes, sterols, fat-soluble vitamins (such as vitamins A, D, E, and K), monoglycerides, diglycerides, triglycerides, phospholipids, and others. “Lipid” may also refer to ethoxylated fatty alcohols such as oleth-10 and laureth-10 and mixtures of ethoxylated mono and diglycerides such as PEG-16 macadamia glycerides and PEG-10 sunflower glycerides. The compounds are hydrophobic or amphiphilic small molecules. The amphiphilic nature of some lipids allows them to form structures such as vesicles, liposomes, or membranes in an aqueous environment. Biological lipids originate entirely or in part from two distinct types of biochemical subunits or “building-blocks”: ketoacyl and isoprene groups. Using this approach, lipids may be divided into eight categories: fatty acids, glycerolipids, glycerophospholipids, sphingolipids, saccharolipids, and polyketides (derived from condensation of ketoacyl subunits); and sterol lipids and prenol lipids (derived from condensation of isoprene subunits). Although the term lipid is sometimes used as a synonym for fats, fats are a subgroup of lipids called triglycerides. Lipids also encompass molecules such as fatty acids and their derivatives (including tri-, di-, monoglycerides, and phospholipids), as well as other sterol-containing metabolites such as cholesterol. Suitable lipids include, e.g., almond oil, argan oil, avocado oil, canola oil, cashew oil, castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, marula oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, Shea butter, soybean oil, sunflower oil, walnut oil, and watermelon seed oil.

The term “humectant” refers to a substance used to keep the slurry and/or oral dissolvable film moist. A humectant attracts and retains the moisture in the air nearby via absorption, drawing the water vapor into or beneath the oral dissolvable film's surface. This is the opposite use of a hygroscopic material where it is used as a desiccant used to draw moisture away. Humectants can be used in oral dissolvable films to increase the solubility of active ingredients, increasing the active ingredients' ability to penetrate a mucosal surface, or its activity time. Examples include, e.g., propylene glycol, hexylene glycol, butylene glycol, aloe vera gel, alpha hydroxy acids (e.g., lactic acid), glyceryl triacetate, and sugar alcohols or polyols (e.g., glycerol, sorbitol, xylitol, and maltitol).

The term “lubricant” or “glidant” refers to a substance added to the formulation (e.g., slurry) to improve processing characteristics. For example, the lubricant can enhance flow of the slurry by reducing interparticulate friction. Suitable lubricants include, e.g., magnesium stearate, calcium stearate, stearic acid, hydrogenated vegetable oil (e.g., Sterotex, Lubritab, and Cutina), mineral oil, polyethylene glycol 4000-6000 (PEG), sodium lauryl sulfate (SLS), sodium hyaluronate, sucrose esters, glyceryl behenate (stelliesters), dimethyl phthalate, diethyl phthalate, dibutyl phthalate, tributyl citrate, triethyl citrate, acetyl citrate, triacetin, dioctyl adipate, diethyl adipate, di(2-methylethyl) adipate, dihexyl adipate, partial fatty acid esters of sugars, polyethylene glycol fatty acid esters, polyethylene glycol fatty alcohol ethers, polyethylene glycol sorbitan fatty acid esters, 2-ethoxy ethanol, ethyl alcohol, propyl alcohol, butyl alcohol, pentyl alcohol, hexyl alcohol, heptyl alcohol, octyl alcohol, dibutyl tartrate, castor oil, or any combination thereof.

The term “stabilizer” refers to a substance that is used to prevent degradation of any one of more substances present in the slurry and/or oral dissolvable film. This would include the active ingredient as well as any of the inactive ingredients (e.g., excipients or additives).

The term “antioxidant” refers to a substance that inhibits or prevents oxidation of any one of more substances present in the slurry and/or oral dissolvable film. This would include the active ingredient as well as any of the inactive ingredients (e.g., excipients or additives). Examples of antioxidants include, e.g., ascorbic acid (vitamin C), vitamin A, α-tocopherol (vitamin E), beta-carotene, glutathione, ubiquinol (coenzyme Q), and selenium.

The term “anti-tacking agent” refers to a substance employed to prevent the formation of lumps (caking) of powdered or granulated materials. Use of the anti-tacking agent can result in the ease of flowability of the solid powders used to form the slurry. Crystalline solids often cake by formation of liquid bridge and subsequent fusion of microcrystals. Amorphous materials can cake by glass transitions and changes in viscosity. Polymorphic phase transitions can also induce caking. Examples include, e.g., calcium silicate, calcium carbonate, and magnesium carbonate.

The term “fragrance” (alternatively known as an odorant or aroma compounds) refers to a substance employed to impart a desired smell or odor.

The term “surfactant” refers to a substance that lower the surface tension (or interfacial tension) between two liquids, between a gas and a liquid, or between a liquid and a solid. When present in the slurry and/or oral dissolvable film described herein, the surfactant may act as a detergent, wetting agent, emulsifier, foaming agent, and/or dispersant.

The term “adjuvant” refers to a substance (e.g., pharmacological or immunological agent) that modifies (e.g., increases) the effect or efficacy of the active ingredient.

The term “release modifier” refers to a substance employed to modify the release of active ingredient from the oral dissolvable film and/or to modify the absorption of active ingredient when administered to the subject. The modified drug release can be contrasted to an immediate release (IR), and includes, e.g., an extended release (XR) or delayed release (DR).

The term “permeation enhancer” refers to a substance employed to increase the delivery the active ingredient, when administered in vivo (e.g., orally), across the desired body surface (e.g., oral mucosa, such as buccal, sublingual, mucosa, or gingival; or an intestinal surface), resulting in an increased absorption of the active ingredient.

The term “powder matrix” refers to a coating of solid substance on the surface of the oral dissolvable film. The solid substance is intended to directly contact the external surface(s) of the polymeric matrix (of the oral dissolvable film), but is not intended to be present within the polymeric matrix. As such, the powder coating does not impregnate or penetrate the polymeric matrix to any appreciable and significant degree. The powder matrix is typically administered to the oral dissolvable film to form the applied coating after the oral dissolvable film has been manufactured (e.g., after the cast slurry has been cured). Alternatively, the powder matrix can be administered during the curing (e.g., while the cast slurry is being cured). Either way, the powder matrix can be applied in any desired manner, including sifting, screening, atomization, static, mechanical agitation, etc. For example, the powder matrix can be atomized through a Nordson or similar static spray gun using compressed air. One such gun creates a fine mist spray of powder particles. The gun statically electrically charges the powder particles so they adhere to a surface of the film that is receiving the powder particles. Another process for applying the powder particles is to admix the particles with a liquid carrier to form a particle-liquid solution. The particle-liquid solution is sprayed on the film. The liquid carrier evaporates, leaving the powder particles on the film. The liquid carrier preferably does not cause the powder particles to dissolve in the liquid carrier. Examples of substances that can be present in the powder matrix include, e.g., a binder, saliva stimulating agent, flavoring agent, taste masking agent, coloring agent, lubricant, sweetening agent, surfactant, pH adjusting agent, buffering agent, glidant, anti-tacking agent, permeation enhancer, or any combination thereof.

The term “unit dosage form” refers to an oral dissolvable film sized to the appropriate dimension, such that the individual film contains a desired amount of active ingredient. Prior to sizing to the appropriate dimension (thereby providing the unit dosage form), the dissolvable film can exist in either the unwound form (e.g., sheet) or in the wound form (e.g., bulk roll).

The term “thickness” refers to the distance between opposite sides of the oral dissolvable film. The thickness is the smallest of the three dimensions (length, width, and thickness). The thickness of the film can be measured by a micrometer screw gauge or calibrated digital Vernier Calipers. The thickness can be evaluated at five different locations (four corners and one at center) and in specific embodiments may be significant to ascertain uniformity in the thickness of the film, as this may be directly related to accuracy of dose distribution in the film.

The term “mass” refers to a measurement of how much matter is in an object. Mass is a combination of the total number of atoms, the density of the atoms, and the type of atoms in an object. Mass is usually measured in grams (which is abbreviated as g) or milligrams (which is abbreviated as mg).

The term “drug load” or “load of active ingredient” refers to the amount of active pharmaceutical ingredient present in the oral dissolvable film.

The term “density” refers to the mass per unit volume of an object (e.g., oral dissolvable film). Density is calculated by dividing the mass of an object by the volume of the object. The volume of an object can be stated as cubic centimeters or milliliters as both are equivalent.

The term “loss on drying (LOD)” refers to the loss of weight expressed as percentage w/w resulting from water and/or volatile matter that can be driven off under specified conditions from an object (e.g., oral dissolvable film). In this technique, a sample of material (e.g., oral dissolvable film) is weighed, heated in an oven for an appropriate period, cooled in the dry atmosphere of a desiccator, and then reweighed. The difference in weight is the loss on drying (LOD). For example, the oral dissolvable film can have a loss on drying (LOD) of 10±2 wt. %.

The term “tack” refers to the tenacity with which the oral dissolvable film adheres to an accessory (a piece of paper) that has been pressed into contact with the film.

The term “tensile strength” refers to the maximum stress applied to a point at which the oral dissolvable film specimen breaks. It is calculated by the applied load at rupture divided by the cross-sectional area of oral dissolvable film, as given in the equation below:

Tensile strength=Load at failure×100/Film thickness×Film width

The term “percent elongation” refers to the relative increase in amount in length upon application of stress. When stress is applied on a film sample, it gets stretched. This is referred to as strain. Strain is basically the deformation of film before it gets broken due to stress. It can be measured by using hounsfield universal testing machine. Generally, elongation of the film increases as the plasticizer content increases. It is calculated by the formula:

% Elongation=Increase in length of film×100/Initial length of film

The term “tear resistance” refers to the resistance which a film offers when some load or force is applied on the film specimen. Specifically, it is the maximum force required to tear the specimen. The load mainly applied can be of a very low rate (e.g., 51 mm/min). The unit of tear resistance is Newton or pounds-force.

The term “Young's modulus” or “elastic modulus” refers to the measure of stiffness of a dissolvable film. It is represented as the ratio of applied stress over strain in the region of elastic deformation as follows:

Young's modulus=Slope×100/Film thickness×Cross head speed

Hard and brittle strips demonstrate a high tensile strength and Young's modulus with small elongation.

The term “folding endurance” refers to number of times the film can be folded without breaking or without any visible crack. Folding endurance gives the brittleness of a film. The method followed to determine endurance value is that the film specimen is repeatedly folded at the same place until it breaks or a visible crack is observed. The number of times the film is folded without breaking or without any visible crack is the calculated folding endurance value.

The term “drug content uniformity,” “uniformity of dosage unit” or “CU” refers to the degree of uniformity in the amount of drug substance among dosage units, and unless otherwise specified, is set forth in USP-NF General Chapter <905> Uniformity of Dosage Units.

The term “dissolution” refers to a substance (e.g., oral dissolvable film or matrix of an oral dissolvable film) dissolving or being dissolved to release the API. When placed in the oral cavity, the substance will dissolve in saliva.

The term “disintegration” refers to a substance (e.g., matrix of an oral dissolvable film) breaking up or falling apart. The substance will lose cohesion or strength and can fragment into pieces. When placed in the oral cavity, the substance will break apart in the saliva.

The term “effective amount” is used herein to generally include an amount of active ingredient present in the oral dissolvable film, effective for treating or preventing a disease, disorder, or condition in a subject, as described herein.

The term “treating” with regard to a subject, refers to improving at least one symptom of the subject's disease, disorder, or condition. Treating includes curing, improving, or at least partially ameliorating the disease, disorder, or condition, or any of the symptoms thereof.

The term “subject” is used herein to generally include humans. The subject can particularly include human infants (e.g., up to 3 years old), human children (e.g., 4-11 years old), human adolescents (e.g., 12-17 years old), and human male adults (e.g., at least 18 years old). Unless otherwise specified, the human can be a male or female.

The term “oral administration” refers to a route of administration where a substance is taken through the mouth. Many medications are taken orally because they are intended to have a systemic effect, reaching different parts of the body via the bloodstream.

The term “mucous membrane” (and related “mucosa” and “mucosal surface”) refers to a membrane that lines various cavities in the body or covers those surfaces. It consists of one or more layers of epithelial cells overlying a layer of loose connective tissue. It is mostly of endodermal origin and is continuous with the skin at various body openings such as the eyes, ears, inside the nose, inside the mouth, lip, vagina, the urethral opening and the anus. Some mucous membranes secrete mucus, a thick protective fluid. The function of the membrane is to stop pathogens and dirt from entering the body and to prevent bodily tissues from becoming dehydrated. Mucosal surfaces specifically include, e.g., oral mucosa, tongue, vaginal mucosa, nasal mucosa, and the anal canal.

The term “transmucosal,” as used herein, refers to any route of administration via a mucosal membrane or mucosal surface. Examples include, but are not limited to, buccal, sublingual, nasal, vaginal, and rectal.

The term “buccal administration” refers to a topical route of administration by which a drug held or applied in the buccal area (in the cheek) diffuses through the oral mucosa (tissues which line the mouth) and enters directly into the bloodstream. Buccal administration may provide better bioavailability of some drugs and a more rapid onset of action compared to oral administration because the medication does not pass through the digestive system and thereby avoids first pass metabolism.

The term “buccal space” (also termed the “buccinator space”) refers to a fascial space of the head and neck (sometimes also termed fascial tissue spaces or tissue spaces). It is a potential space in the cheek, and is paired on each side. The buccal space is superficial to the buccinator muscle and deep to the platysma muscle and the skin. The buccal space is part of the subcutaneous space, which is continuous from head to toe.

The term “oral mucosa” refers to the mucous membrane lining the inside of the mouth and consists of stratified squamous epithelium termed oral epithelium and an underlying connective tissue termed lamina propria. Oral mucosa can be divided into three main categories based on function and histology: (1) Masticatory mucosa, keratinized stratified squamous epithelium, found on the dorsum of the tongue, hard palate and attached gingiva; (2) Lining mucosa, nonkeratinized stratified squamous epithelium, found almost everywhere else in the oral cavity, including the: (a) Buccal mucosa refers to the inside lining of the cheeks and floor of the mouth and is part of the lining mucosa; (b) Labial mucosa refers to the inside lining of the lips and is part of the lining mucosa; and (c) Alveolar mucosa refers to the lining between the buccal and labial mucosae. It is a brighter red, smooth and shiny with many blood vessels, and is not connected to underlying tissue by rete pegs; and (3) Specialized mucosa, specifically in the regions of the taste buds on lingual papillae on the dorsal surface of the tongue that contains nerve endings for general sensory reception and taste perception.

The term “sublingual administration,” from the Latin for “under the tongue,” refers to the pharmacological route of administration by which substances diffuse into the blood through tissues under the tongue. When a drug comes in contact with the mucous membrane beneath the tongue, it is absorbed. Because the connective tissue beneath the epithelium contains a profusion of capillaries, the substance then diffuses into them and enters the venous circulation. In contrast, substances absorbed in the intestines are subject to first-pass metabolism in the liver before entering the general circulation. Sublingual administration has certain advantages over oral administration. Being more direct, it is often faster, and it ensures that the substance will risk degradation only by salivary enzymes before entering the bloodstream, whereas orally administered drugs must survive passage through the hostile environment of the gastrointestinal tract, which risks degrading them, by either stomach acid or bile, or by enzymes such as monoamine oxidase (MAO). Furthermore, after absorption from the gastrointestinal tract, such drugs must pass through the liver, where they may be extensively altered; this is known as the first pass effect of drug metabolism. Due to the digestive activity of the stomach and intestines, the oral route is unsuitable for certain substances.

The term “gingival administration” refers to the pharmacological route of administration by which substances diffuse into the blood through tissues in the gums. The gums or gingiva (plural: gingivae), consist of the mucosal tissue that lies over the mandible and maxilla inside the mouth.

The term “enteral administration” refers to a drug administration via the human gastrointestinal tract. Enteral administration involves the esophagus, stomach, and small and large intestines (i.e., the gastrointestinal tract). Methods of administration include oral and rectal. Enteral administration may be divided into three different categories, depending on the entrance point into the GI tract: oral (by mouth), gastric (through the stomach), and rectal (from the rectum). (Gastric introduction involves the use of a tube through the nasal passage (NG tube) or a tube in the belly leading directly to the stomach (PEG tube). Rectal administration usually involves rectal suppositories.) Enteral medications come in various forms, including, e.g., tablets to swallow, chew or dissolve in water; capsules and chewable capsules (with a coating that dissolves in the stomach or bowel to release the medication there), oral soluble films, time-release or sustained-release tablets and capsules (which release the medication gradually), osmotic delivery systems, powders or granules, and liquid medications or syrups.

### Specific Ranges, Values, and Embodiments

The specific embodiments describing the ranges and values provided below are for illustration purposes only, and do not otherwise limit the scope of the disclosed subject matter, as defined by the claims.

In specific embodiments, the psychedelic compound selected from the group consisting of psilocybin, psilocin, baeocystin, mescaline, LSD, ketamine, salvinorin A, ibotenic acid, muscimol, DMT, MDMA, MDEA, MDA, and combinations thereof.

In specific embodiments, the psychedelic compound is at least one of psilocybin, psilocin and baeocystin.

In specific embodiments, the psychedelic compound is obtained from the genera Copelandia, Gymnopilus, Inocybe, Mycena, Panaeolus, Pholiotina, Pluteus, or Psilocybe.

In specific embodiments, the psychedelic compound is present as an extract obtained from the genera Copelandia, Gymnopilus, Inocybe, Mycena, Panaeolus, Pholiotina, Pluteus, or Psilocybe.

In specific embodiments, the psychedelic compound is present as a purified extract obtained from the genera Copelandia, Gymnopilus, Inocybe, Mycena, Panaeolus, Pholiotina, Pluteus, or Psilocybe.

In specific embodiments, the psychedelic compound is at least one of psilocybin, psilocin and baeocystin, obtained from the genera Copelandia, Gymnopilus, Inocybe, Mycena, Panaeolus, Pholiotina, Pluteus, or Psilocybe.

In specific embodiments, the psychedelic compound is at least one of psilocybin, psilocin and baeocystin, obtained as an extract from mushrooms.

In specific embodiments, the psychedelic compound is at least one of psilocybin, psilocin and baeocystin, synthetically prepared.

In specific embodiments, the psychedelic compound is present in up to 200 mg.

In specific embodiments, the psychedelic compound is present in up to 150 mg.

In specific embodiments, the psychedelic compound is present in up to 100 mg.

In specific embodiments, the psychedelic compound is present in up to 50 mg.

In specific embodiments, the psychedelic compound is present in up to 25 mg.

In specific embodiments, the psychedelic compound is present in up to 10 mg.

In specific embodiments, the psychedelic compound is present in up to 5 mg.

In specific embodiments, the psychedelic compound is present in up to 2.5 mg.

In specific embodiments, the psychedelic compound is present in up to 1 mg.

In specific embodiments, the psychedelic compound is present in up to 0.5 mg.

In specific embodiments, the psychedelic compound is present in up to 0.25 mg.

In specific embodiments, the psychedelic compound is present in 1-200 mg.

In specific embodiments, the psychedelic compound is present in 1-150 mg.

In specific embodiments, the psychedelic compound is present in 1-100 mg.

In specific embodiments, the psychedelic compound is present in 1-50 mg.

In specific embodiments, the psychedelic compound is present in 1-25 mg.

In specific embodiments, the psychedelic compound is present in 1-10 mg.

In specific embodiments, the psychedelic compound is present in 0.01-5 mg.

In specific embodiments, the psychedelic compound is present in 0.01-2.5 mg.

In specific embodiments, the psychedelic compound is present in 0.01-1 mg.

In specific embodiments, the psychedelic compound is present in 0.01-0.5 mg.

In specific embodiments, the psychedelic compound is present in 0.01-0.25 mg.

In specific embodiments, the psychedelic compound is present in up to 35 wt. % of the oral dissolvable film.

In specific embodiments, the psychedelic compound is present in up to 30 wt. % of the oral dissolvable film.

In specific embodiments, the psychedelic compound is present in up to 25 wt. % of the oral dissolvable film.

In specific embodiments, the psychedelic compound is present in up to 20 wt. % of the oral dissolvable film.

In specific embodiments, the psychedelic compound is present in up to 15 wt. % of the oral dissolvable film.

In specific embodiments, the psychedelic compound is present in up to 10 wt. % of the oral dissolvable film.

In specific embodiments, the psychedelic compound is present in up to 5 wt. % of the oral dissolvable film.

In specific embodiments, the psychedelic compound is present in 0.01-15 wt. % of the oral dissolvable film.

In specific embodiments, the psychedelic compound is present in 0.01-10 wt. % of the oral dissolvable film.

In specific embodiments, the psychedelic compound is present in 0.01-5 wt. % of the oral dissolvable film.

In specific embodiments, the psychedelic compound is present in 0.01-3.5 wt. % of the oral dissolvable film.

In specific embodiments, the psychedelic compound is present in 0.01-2.5 wt. % of the oral dissolvable film.

In specific embodiments, the psychedelic compound is present in 0.01-1.5 wt. % of the oral dissolvable film.

In specific embodiments, the psychedelic compound is present in 0.01-1 wt. % of the oral dissolvable film.

In specific embodiments, the psychedelic compound is present in 0.01-0.5 wt. % of the oral dissolvable film.

In specific embodiments, the psychedelic compound is present in 0.01-0.25 wt. % of the oral dissolvable film.

In specific embodiments, the psychedelic compound is present in 25±5 mg/cm² of the oral dissolvable film.

In specific embodiments, the psychedelic compound is present in 50±10 mg/cm² of the oral dissolvable film.

In specific embodiments, the psychedelic compound is present in 20±4 mg/cm² of the oral dissolvable film.

In specific embodiments, the psychedelic compound is present in 15±3 mg/cm² of the oral dissolvable film.

In specific embodiments, the psychedelic compound is present in 10±2 mg/cm² of the oral dissolvable film.

In specific embodiments, the psychedelic compound is present in 5±1 mg/cm² of the oral dissolvable film.

In specific embodiments, the psychedelic compound is present in 2.5±0.5 mg/cm² of the oral dissolvable film.

In specific embodiments, the psychedelic compound is present in 2±0.5 mg/cm² of the oral dissolvable film.

In specific embodiments, the psychedelic compound is present in 1±0.25 mg/cm² of the oral dissolvable film.

In specific embodiments, the psychedelic compound is present in 0.5±0.1 mg/cm² of the oral dissolvable film.

In specific embodiments, the psychedelic compound has a purity of at least 95 wt. % pure.

In specific embodiments, the psychedelic compound has a purity of at least 97.5 wt. % pure.

In specific embodiments, the psychedelic compound has a purity of at least 98 wt. % pure.

In specific embodiments, the psychedelic compound has a purity of at least 99 wt. % pure.

In specific embodiments, the psychedelic compound has a purity of at least 99.5 wt. % pure.

In specific embodiments, the oral dissolvable film contains up to about 15 wt. % water or moisture.

In specific embodiments, the oral dissolvable film contains up to about 15 wt. % residual solvent.

In specific embodiments, the oral dissolvable film contains up to about 12 wt. % water or moisture.

In specific embodiments, the oral dissolvable film contains up to about 12 wt. % residual solvent.

In specific embodiments, the oral dissolvable film contains 9±5 wt. % water or moisture.

In specific embodiments, the oral dissolvable film contains 9±5 wt. % residual solvent.

In specific embodiments, the oral dissolvable film contains 8±5 wt. % water or moisture.

In specific embodiments, the oral dissolvable film contains 8±5 wt. % residual solvent.

In specific embodiments, the oral dissolvable film contains 8±4 wt. % water or moisture.

In specific embodiments, the oral dissolvable film contains 8±4 wt. % residual solvent.

In specific embodiments, the oral dissolvable film contains 8±3 wt. % water or moisture.

In specific embodiments, the oral dissolvable film contains 8±3 wt. % residual solvent.

In specific embodiments, the oral dissolvable film has a mass of 300±100 mg.

In specific embodiments, the oral dissolvable film has a mass of 300±60 mg.

In specific embodiments, the oral dissolvable film has a mass of 250±75 mg.

In specific embodiments, the oral dissolvable film has a mass of 250±50 mg.

In specific embodiments, the oral dissolvable film has a mass of 225±50 mg.

In specific embodiments, the oral dissolvable film has a mass of 200±40 mg.

In specific embodiments, the oral dissolvable film has a mass of 200±30 mg.

In specific embodiments, the oral dissolvable film has a mass of 175±35 mg.

In specific embodiments, the oral dissolvable film has a mass of 175±25 mg.

In specific embodiments, the oral dissolvable film has a mass of 150±30 mg.

In specific embodiments, the oral dissolvable film has a mass of 150±20 mg.

In specific embodiments, the oral dissolvable film has a mass of 125±25 mg.

In specific embodiments, the oral dissolvable film has a mass of 125±15 mg.

In specific embodiments, the oral dissolvable film has a mass of 100±20 mg.

In specific embodiments, the oral dissolvable film has a mass of 100±10 mg.

In specific embodiments, the oral dissolvable film has a mass of 75±15 mg.

In specific embodiments, the oral dissolvable film has a mass of 75±7.5 mg.

In specific embodiments, the oral dissolvable film has the following dimensions: 44±6 mm×22±3 mm×0.12±0.02 mm.

In specific embodiments, the oral dissolvable film has a thickness of less than 0.35±0.15 mm.

In specific embodiments, the oral dissolvable film has a thickness of less than 0.35±0.10 mm.

In specific embodiments, the oral dissolvable film has a thickness of less than 0.35±0.05 mm.

In specific embodiments, the oral dissolvable film has a thickness of less than 0.25±0.10 mm.

In specific embodiments, the oral dissolvable film has a thickness of less than 0.25±0.075 mm.

In specific embodiments, the oral dissolvable film has a thickness of less than 0.25±0.05 mm.

In specific embodiments, the oral dissolvable film comprises less than 10 wt. % variance of psychedelic compound, per unit area of the oral dissolvable film.

In specific embodiments, the oral dissolvable film comprises less than 7.5 wt. % variance of psychedelic compound, per unit area of the oral dissolvable film.

In specific embodiments, the oral dissolvable film comprises less than 5 wt. % variance of psychedelic compound, per unit area of the oral dissolvable film.

In specific embodiments, the oral dissolvable film comprises less than 2.5 wt. % variance of psychedelic compound, per unit area of the oral dissolvable film.

In specific embodiments, the oral dissolvable film comprises less than 1 wt. % variance of psychedelic compound, per unit area of the oral dissolvable film.

In specific embodiments, the oral dissolvable film has a content uniformity, such that among two or more samples, the amount of psychedelic compound ranges from 85% to 115%, with the standard deviation of less than or equal to 6%.

In specific embodiments, the oral dissolvable film has a content uniformity, such that among two or more samples, the amount of psychedelic compound ranges from 85% to 115%, with the standard deviation of less than or equal to 5%.

In specific embodiments, the oral dissolvable film has a content uniformity, such that among two or more samples, the amount of psychedelic compound ranges from 90% to 110%, with the standard deviation of less than or equal to 6%.

In specific embodiments, the oral dissolvable film has a content uniformity, such that among two or more samples, the amount of psychedelic compound ranges from 90% to 110%, with the standard deviation of less than or equal to 5%.

In specific embodiments, the flowable water-soluble or water swellable film-forming matrix includes each of plasticizer, binder, preservative, and solvent.

In specific embodiments, the flowable water-soluble or water swellable film-forming matrix includes at least one of coloring agent, flavoring agent, sweetening agent, filler, bulking agent, saliva stimulating agent, stabilizing and thickening agent, gelling agent, taste masking agent, pigment, lubricant, release modifier, adjuvant, solubilizer & emulsifier, fragrance, emulsifier, surfactant, pH adjusting agent, buffering agent, lipid, glidant, stabilizer, antioxidant, anti-tacking agent, and humectant.

In specific embodiments, the flowable water-soluble or water swellable film-forming matrix includes each of plasticizer, binder, preservative, and solvent; and further includes at least one of coloring agent, flavoring agent, sweetening agent, filler, bulking agent, saliva stimulating agent, stabilizing and thickening agent, gelling agent, taste masking agent, pigment, lubricant, release modifier, adjuvant, solubilizer & emulsifier, fragrance, emulsifier, surfactant, pH adjusting agent, buffering agent, lipid, glidant, stabilizer, antioxidant, anti-tacking agent, and humectant.

In specific embodiments, the oral dissolvable film includes a flowable water-soluble or water swellable film-forming matrix that includes a polymer comprises each of plasticizer, binder, preservative, and solvent.

In specific embodiments, the flowable water-soluble or water swellable film-forming matrix that includes a polymer further includes at least one of coloring agent, flavoring agent, sweetening agent, filler, bulking agent, saliva stimulating agent, stabilizing and thickening agent, gelling agent, taste masking agent, pigment, lubricant, release modifier, adjuvant, solubilizer & emulsifier, fragrance, emulsifier, surfactant, pH adjusting agent, buffering agent, lipid, glidant, stabilizer, antioxidant, anti-tacking agent, and humectant.

In specific embodiments, the oral dissolvable film includes: (a) plasticizer, (b) solvent, (c) sweetener, (d) flavoring agent, (e) binder, (f) coloring agent, (g) preservative, and (h) psychedelic compound selected from the group consisting of psilocybin, psilocin, baeocystin, mescaline, LSD, ketamine, salvinorin A, ibotenic acid, muscimol, DMT, MDMA, MDEA, MDA, and combinations thereof.

In specific embodiments, the oral dissolvable film includes: (a) plasticizer selected from the group consisting of glycerol, glycerol monoacetate, diacetate or triacetate, triacetin, polysorbate, cetyl alcohol, propylene glycol, sorbitol, sodium diethylsulfosuccinate, triethyl citrate, and tributyl citrate, (b) solvent selected from the group consisting of water, ethanol, and combinations thereof, (c) sweetener, (d) flavoring agent, (e) binder selected from the group consisting of pectin, pullulan, starch, pregelatinized starch, gelatin, polyvinylpyrrolidone, methylcellulose, sodium carboxymethylcellulose, ethylcellulose, polyacrylamides, polyvinyloxoazolidone, and polyvinylalcohols, (f) coloring agent, (g) preservative selected from the group consisting of benzoate salt, sorbate salt, natamycin, and combinations thereof, and (h) psychedelic compound selected from the group consisting of psilocybin, psilocin, baeocystin, mescaline, LSD, ketamine, salvinorin A, ibotenic acid, muscimol, DMT, MDMA, MDEA, MDA, and combinations thereof.

In specific embodiments, the oral dissolvable film includes: (a) 10±5 wt. % plasticizer, (b) 8±5 wt. % solvent, (c) 10±5 wt. % sweetener, (d) 8±5 wt. % flavoring agent, (e) 25±10 wt. % binder, (f) 0.02±0.01 wt. % coloring agent, (g) 0.02±0.01 wt. % preservative, and (h) 17±16.5 wt. % psychedelic compound selected from the group consisting of psilocybin, psilocin, baeocystin, mescaline, LSD, ketamine, salvinorin A, ibotenic acid, muscimol, DMT, MDMA, MDEA, MDA, and combinations thereof.

In specific embodiments, the oral dissolvable film is bioerodible.

In specific embodiments, the oral dissolvable film is mucoadhesive.

In specific embodiments, the sweetener includes one or more of sucralose, acesulfame potassium, and stevia.

In specific embodiments, the psychedelic compound is encapsulated.

In specific embodiments, the psychedelic compound is encapsulated in the form of liposomes, micelles, or both.

In specific embodiments, the oral dissolvable film is configured to dissolve in the oral cavity within 180 seconds.

In specific embodiments, the oral dissolvable film is configured to dissolve in the oral cavity within 120 seconds.

In specific embodiments, the oral dissolvable film is configured to dissolve in the oral cavity within 60 seconds.

In specific embodiments, the oral dissolvable film is configured to dissolve in the oral cavity within 30 seconds.

In specific embodiments, the oral dissolvable film is configured to dissolve in the oral cavity in 10-180 seconds.

In specific embodiments, the oral dissolvable film is configured to dissolve in the oral cavity in 10-150 seconds.

In specific embodiments, the oral dissolvable film is configured to dissolve in the oral cavity in 10-120 seconds.

In specific embodiments, the oral dissolvable film is configured to dissolve in the oral cavity in 10-90 seconds.

In specific embodiments, the oral dissolvable film is configured to dissolve in the oral cavity in 10-60 seconds.

In specific embodiments, the oral dissolvable film is configured to dissolve in the oral cavity in 20-180 seconds.

In specific embodiments, the oral dissolvable film is configured to dissolve in the oral cavity in 20-150 seconds.

In specific embodiments, the oral dissolvable film is configured to dissolve in the oral cavity in 20-120 seconds.

In specific embodiments, the oral dissolvable film is configured to dissolve in the oral cavity in 20-90 seconds.

In specific embodiments, the oral dissolvable film is configured to dissolve in the oral cavity in 20-60 seconds.

In specific embodiments, the oral dissolvable film is configured to dissolve in the oral cavity in 30±10 seconds.

In specific embodiments, the oral dissolvable film is configured to dissolve in the oral cavity in 30±5 seconds.

In specific embodiments, the oral dissolvable film is administered to a subject to treat a disease or disorder ameliorated by a psychedelic compound.

In specific embodiments, treating the disease or disorder includes preventing the disease or disorder from occurring.

In specific embodiments, treating the disease or disorder includes curing the disease or disorder.

In specific embodiments, treating the disease or disorder includes healing the disease or disorder.

In specific embodiments, treating the disease or disorder includes alleviating the disease or disorder.

In specific embodiments, treating the disease or disorder includes alleviating one or more symptoms of the disease or disorder.

In specific embodiments, treating the disease or disorder includes relieving the disease or disorder.

In specific embodiments, treating the disease or disorder includes relieving one or more symptoms of the disease or disorder.

In specific embodiments, treating the disease or disorder includes altering the disease or disorder.

In specific embodiments, treating the disease or disorder includes altering one or more symptoms of the disease or disorder.

In specific embodiments, treating the disease or disorder includes remedying the disease or disorder.

In specific embodiments, treating the disease or disorder includes remedying one or more symptoms of the disease or disorder.

In specific embodiments, treating the disease or disorder includes ameliorating the disease or disorder.

In specific embodiments, treating the disease or disorder includes ameliorating one or more symptoms of the disease or disorder.

In specific embodiments, treating the disease or disorder includes improving the disease or disorder.

In specific embodiments, treating the disease or disorder includes improving one or more symptoms of the disease or disorder.

In specific embodiments, treating the disease or disorder includes stabilizing or affecting the disease or disorder.

In specific embodiments, treating the disease or disorder includes stabilizing or affecting one or more symptoms of the disease or disorder.

In specific embodiments, treating the disease or disorder includes a psychological or neurological disorder.

In specific embodiments, treating the disease or disorder includes treating obsessive compulsive disorder (OCD).

In specific embodiments, treating the disease or disorder includes treating depression.

In specific embodiments, treating the disease or disorder includes treating pain.

In specific embodiments, treating the disease or disorder includes treating irritability.

In specific embodiments, treating the disease or disorder includes treating fibromyalgia.

In specific embodiments, treating the disease or disorder includes treating post-traumatic stress disorder (PTSD).

In specific embodiments, treating the disease or disorder includes treating cluster headaches.

In specific embodiments, treating the disease or disorder includes treating paranoia.

In specific embodiments, treating the disease or disorder includes treating psychosis.

In specific embodiments, treating the disease or disorder includes treating anxiety.

In specific embodiments, treating the disease or disorder includes treating panic attacks.

In specific embodiments, treating the disease or disorder includes treating flashbacks.

In specific embodiments, treating the disease or disorder includes treating smoking addiction.

In specific embodiments, treating the disease or disorder includes treating alcohol addiction.

In specific embodiments, treating the disease or disorder includes treating cocaine addiction.

In specific embodiments, treating the disease or disorder includes treating a drug addiction.

In specific embodiments, treating the disease or disorder includes alleviating one or more symptoms of obsessive compulsive disorder (OCD).

In specific embodiments, treating the disease or disorder includes alleviating one or more symptoms of depression.

In specific embodiments, treating the disease or disorder includes alleviating one or more symptoms of pain.

In specific embodiments, treating the disease or disorder includes alleviating one or more symptoms of irritability.

In specific embodiments, treating the disease or disorder includes alleviating one or more symptoms of fibromyalgia.

In specific embodiments, treating the disease or disorder includes alleviating one or more symptoms of post-traumatic stress disorder (PTSD).

In specific embodiments, treating the disease or disorder includes alleviating one or more symptoms of cluster headaches.

In specific embodiments, treating the disease or disorder includes alleviating one or more symptoms of paranoia.

In specific embodiments, treating the disease or disorder includes alleviating one or more symptoms of psychosis.

In specific embodiments, treating the disease or disorder includes alleviating one or more symptoms of anxiety.

In specific embodiments, treating the disease or disorder includes alleviating one or more symptoms of panic attacks.

In specific embodiments, treating the disease or disorder includes alleviating one or more symptoms of flashbacks.

In specific embodiments, treating the disease or disorder includes alleviating one or more symptoms of smoking addiction.

In specific embodiments, treating the disease or disorder includes alleviating one or more symptoms of alcohol addiction.

In specific embodiments, treating the disease or disorder includes alleviating one or more symptoms of cocaine addiction.

In specific embodiments, treating the disease or disorder includes alleviating one or more symptoms of drug addiction.

In specific embodiments, the oral dissolvable film is administered to the subject to improve creativity.

In specific embodiments, the oral dissolvable film is administered to the subject to boost physical energy level.

In specific embodiments, the oral dissolvable film is administered to the subject to attain emotional balance.

In specific embodiments, the oral dissolvable film is administered to the subject to increase performance on problem-solving tasks.

In specific embodiments, the oral dissolvable film is administered to the subject to increase emotional well-being.

In specific embodiments, the oral dissolvable film is administered to the subject to improve the subject's mood.

In specific embodiments, the psychedelic compound is administered to the subject in a low dose.

In specific embodiments, the psychedelic compound is administered to the subject in a microdose.

In specific embodiments, a low dose of the psychedelic compound is administered to the subject, such that the low dose is sub-threshold or sub-therapeutic, insufficient to produce whole-body effects, but is high enough to allow the cellular response to be observed.

In specific embodiments, a microdose of the psychedelic compound is administered to the subject, such that the microdose is sub-threshold or sub-therapeutic, insufficient to produce whole-body effects, but is high enough to allow the cellular response to be observed.

In specific embodiments, 1-10 oral dissolvable films a day are administered to the subject.

In specific embodiments, 1-8 oral dissolvable films a day are administered to the subject.

In specific embodiments, 1-6 oral dissolvable films a day are administered to the subject.

In specific embodiments, 1-4 oral dissolvable films a day are administered to the subject.

In specific embodiments, 1-2 oral dissolvable films a day are administered to the subject.

In specific embodiments, the dissolvable film is administered orally (PO).

In specific embodiments, the dissolvable film is placed within the oral cavity of the patient.

In specific embodiments, the dissolvable film is placed on the top of the tongue of the patient.

In specific embodiments, the dissolvable film is placed under the tongue of the patient.

In specific embodiments, the dissolvable film is placed on the inside of the cheek of the patient.

In specific embodiments, the dissolvable film is placed on the gums of the patient.

In specific embodiments, the dissolvable film is placed between the gums and lips of the patient.

In specific embodiments, the psychedelic compound is delivered enterally, transmucosally, buccally, or sublingually.

In specific embodiments, the psychedelic compound is delivered enterally.

In specific embodiments, the psychedelic compound is delivered transmucosally.

In specific embodiments, the psychedelic compound is delivered buccally.

In specific embodiments, the psychedelic compound is delivered sublingually.

### ENUMERATED EMBODIMENTS

Specific enumerated embodiments <1.> to <44.> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

<1.> An oral dissolvable film comprising:
(i) a flowable water-soluble or water swellable film-forming matrix that includes a polymer, and
(ii) psychedelic compound selected from the group consisting of psilocybin, psilocin, baeocystin, mescaline, LSD, ketamine, salvinorin A, ibotenic acid, muscimol, DMT, MDMA, MDEA, MDA, and combinations thereof.

<2.> The oral dissolvable film of embodiment <1.>, wherein the psychedelic compound is at least one of synthetic psilocybin, synthetic psilocin and synthetic baeocystin.

<3.> The oral dissolvable film of embodiment <1.>, wherein the psychedelic compound is at least one of psilocybin, psilocin and baeocystin, each obtained from the genera Copelandia, Gymnopilus, Inocybe, Mycena, Panaeolus, Pholiotina, Pluteus, or Psilocybe.

<4.> The oral dissolvable film of any one of the above embodiments, wherein the psychedelic compound is present in up to 250 mg.

<5.> The oral dissolvable film of any one of the above embodiments, wherein the psychedelic compound is present in 0.01 to 250 mg.

<6.> The oral dissolvable film of any one of the above embodiments, wherein the psychedelic compound is at least one of psilocybin, psilocin and baeocystin, present in a combined amount of up to 100 mg.

<7.> The oral dissolvable film of any one of the above embodiments, wherein the psychedelic compound is at least one of psilocybin, psilocin and baeocystin, present in a combined amount of up to 50 mg.

<8.> The oral dissolvable film of any one of the above embodiments, wherein the psychedelic compound is at least one of psilocybin, psilocin and baeocystin, present in a combined amount of up to 10 mg.

<9.> The oral dissolvable film of any one of the above embodiments, wherein the psychedelic compound is at least one of psilocybin, psilocin and baeocystin, present in a combined amount of up to 5 mg.

<10.> The oral dissolvable film of any one of the above embodiments, wherein the psychedelic compound is at least one of psilocybin, psilocin and baeocystin, present in a combined amount of up to 1 mg.

<11.> The oral dissolvable film of any one of the above embodiments, wherein the psychedelic compound is at least one of psilocybin, psilocin and baeocystin, present in a combined amount of 0.01 to 5 mg.

<12.> The oral dissolvable film of any one of the above embodiments, wherein the psychedelic compound is at least one of psilocybin, psilocin and baeocystin, present in a combined amount of 0.01 to 2.5 mg.

<13.> The oral dissolvable film of any one of the above embodiments, wherein the psychedelic compound is at least one of psilocybin, psilocin and baeocystin, present in a combined amount of 0.01 to 1 mg.

<14.> The oral dissolvable film of any one of the above embodiments, wherein the psychedelic compound is at least one of psilocybin, psilocin and baeocystin, present in a combined amount of 0.01 to 0.5 mg.

<15.> The oral dissolvable film of any one of the above embodiments, wherein the psychedelic compound is at least one of psilocybin, psilocin and baeocystin, present in a combined amount of 0.01 to 0.1 mg.

<16.> The oral dissolvable film of any one of the above embodiments, wherein the psychedelic compound has a purity of at least 90 wt. % pure.

<17.> The oral dissolvable film of any one of the above embodiments, wherein the psychedelic compound has a purity of at least 95 wt. % pure.

<18.> The oral dissolvable film of any one of the above embodiments, wherein the psychedelic compound has a purity of at least 97.5 wt. % pure.

<19.> The oral dissolvable film of any one of the above embodiments, wherein the psychedelic compound has a purity of at least 99 wt. % pure.

<20.> The oral dissolvable film of any one of the above embodiments, wherein the psychedelic compound has a purity of at least 99.5 wt. % pure.

<21.> The oral dissolvable film of any one of the above embodiments, having a content uniformity, such that among two or more samples, the amount of psychedelic compound ranges from 85% to 115%, with the standard deviation of less than or equal to 6%.

<22.> The oral dissolvable film of any one of the above embodiments, that includes the psychedelic compound in 2.5±2.3 mg/cm².

<23.> The oral dissolvable film of any one of the above embodiments, that includes the psychedelic compound in 25±5 mg/cm².

<24.> The oral dissolvable film of any one of the above embodiments, having a mass of 200±50 mg.

<25.> The oral dissolvable film of any one of the above embodiments, having a mass of 150±25 mg.

<26.> The oral dissolvable film of any one of the above embodiments, having a thickness of less than 0.350 mm.

<27.> The oral dissolvable film of any one of the above embodiments, having a content uniformity, such that among two or more samples, the amount of psychedelic compound ranges from 85% to 115%, with the standard deviation of less than or equal to 6%.

<28.> The oral dissolvable film of any one of the above embodiments, exhibiting a high stability such that at least 90 wt. % of the psychedelic compound remains in the oral dissolvable film, under accelerated stability conditions of ≥40° C., relative humidity (RH) 75±5%, over a period of time of ≥3 months.

<29.> The oral dissolvable film of any one of the above embodiments, exhibiting a high stability such that at least 95 wt. % of the psychedelic compound remains in the oral dissolvable film, under accelerated stability conditions of ≥40° C., relative humidity (RH) 75±5%, over a period of time of ≥3 months.

<30.> The oral dissolvable film of any one of the above embodiments, exhibiting a high stability such that at least 97.5 wt. % of the psychedelic compound remains in the oral dissolvable film, under accelerated stability conditions of ≥40° C., relative humidity (RH) 75±5%, over a period of time of ≥3 months.

<31.> The oral dissolvable film of any one of the above embodiments, exhibiting a high stability such that at least 99 wt. % of the psychedelic compound remains in the oral dissolvable film, under accelerated stability conditions of ≥40° C., relative humidity (RH) 75±5%, over a period of time of ≥3 months.

<32.> The oral dissolvable film of any one of the above embodiments, wherein the flowable water-soluble or water swellable film-forming matrix that includes a polymer includes each of plasticizer, binder, preservative, and solvent.

<33.> The oral dissolvable film of any one of the above embodiments, wherein the flowable water-soluble or water swellable film-forming matrix that includes a polymer further includes at least one of coloring agent, flavoring agent, sweetening agent, filler, bulking agent, saliva stimulating agent, stabilizing and thickening agent, gelling agent, taste masking agent, pigment, lubricant, release modifier, adjuvant, solubilizer & emulsifier, fragrance, emulsifier, surfactant, pH adjusting agent, buffering agent, lipid, glidant, stabilizer, antioxidant, anti-tacking agent, and humectant.

<34.> The oral dissolvable film of any one of the above embodiments, configured to dissolve in the oral cavity within 120 seconds.

<35.> The oral dissolvable film of any one of the above embodiments, that includes:
(a) plasticizer,
(b) solvent,
(c) sweetener,
(d) flavoring agent,
(e) binder,
(f) coloring agent,
(g) preservative, and
(h) psychedelic compound selected from the group consisting of psilocybin, psilocin, baeocystin, mescaline, LSD, ketamine, salvinorin A, ibotenic acid, muscimol, DMT, MDMA, MDEA, MDA, and combinations thereof.

<36.> A method of treating in a subject a disease or disorder ameliorated by a psychedelic compound, the method comprising orally administering to the subject an oral dissolvable film of any one embodiments <1.> to <35.>, in an amount and for a period of time sufficient to effectively treat the disease or disorder.

<37.> A method of treating in a subject a psychological or neurological disorder, the method comprising orally administering to the subject an oral dissolvable film of any one embodiments <1.> to <35.>, in an amount and for a period of time sufficient to effectively treat the psychological or neurological disorder.

<38.> The method of any one of embodiments <36.> to <37.>, wherein the psychological or neurological disorder comprises at least one of obsessive compulsive disorder (OCD), depression, pain, irritability, fibromyalgia, post-traumatic stress disorder (PTSD), cluster headaches, paranoia, psychosis, anxiety, panic attacks, flashbacks, smoking addiction, alcohol addiction, and cocaine addiction.

<39.> A method comprising orally administering to a subject an oral dissolvable film of any one embodiments <1.> to <35.>, wherein the oral dissolvable film comprises a low dose or microdose of the psychedelic compound.

<40.> The method of embodiment <39.>, wherein the low dose or microdose of the psychedelic compound is sub-threshold or sub-therapeutic insufficient to produce whole-body effects, but is high enough to allow the cellular response to be observed.

<41.> The method of any one of embodiments <39.> to <40.>, which is a method to improve creativity, boost physical energy level, attain emotional balance, increase performance on problem-solving tasks, to treat anxiety, to treat depression, to treat addiction, or any combination thereof.

<42.> The method of any one of embodiments <39.> to <41.>, which is a method to treat at least one of obsessive compulsive disorder (OCD), pain, irritability, fibromyalgia, post-traumatic stress disorder (PTSD), cluster headaches, paranoia, psychosis, anxiety, panic attacks, flashbacks, smoking addiction, alcohol addiction, and cocaine addiction.

<43.> The method of any one of embodiments <36.> to <42.>, wherein 1-5 oral dissolvable films are orally administered a day.

<44.> The method of any one of embodiments <36.> to <43.>, wherein the psychedelic compound is delivered enterally, transmucosally, or sublingually.

1. A method comprising orally administering to a subject an oral dissolvable film comprising:
(i) a flowable water-soluble or water swellable film-forming matrix that includes a polymer, and
(ii) psilocybin, psilocin, baeocystin, or a combination thereof;
wherein,
the psilocybin, psilocin, baeocystin, or combination thereof is present in a combined amount of 0.01 to 5 mg;
the psilocybin, psilocin, baeocystin, or combination thereof has a purity of at least 99 wt. % pure; and
the psilocybin, psilocin, baeocystin, or combination thereof is obtained from the genera Copelandia, Gymnopilus, Inocybe, Mycena, Panaeolus, Pholiotina, Pluteus, or Psilocybe.

2. The method of claim 1, comprising improving creativity, boosting physical energy level, attaining emotional balance, increasing performance on problem-solving tasks, treating anxiety, treating depression, treating addiction, or any combination thereof.

3. The method of claim 1, comprising treating at least one of obsessive compulsive disorder (OCD), pain, irritability, fibromyalgia, post-traumatic stress disorder (PTSD), cluster headaches, paranoia, psychosis, anxiety, panic attacks, flashbacks, smoking addiction, alcohol addiction, and cocaine addiction.

4. The method of claim 1, wherein 1-5 oral dissolvable films are orally administered a day.

5. The method of claim 1, wherein the psilocybin, psilocin, baeocystin, or combination thereof is delivered enterally, sublingually, or transmucosally.

6. The method of claim 1, wherein the oral dissolvable film comprises the psilocybin, psilocin, baeocystin, or combination in a combined amount of 0.05 to 2.5 mg.

7. The method of claim 1, wherein the oral dissolvable film comprises the psilocybin, psilocin, baeocystin, or combination thereof in a combined amount of 0.05 to 1 mg.

8. The method of claim 1, wherein the oral dissolvable film comprises the psilocybin, psilocin, baeocystin, or combination thereof in a combined amount of 0.1 to 1 mg.

9. The method of claim 1, wherein the oral dissolvable film comprises the psilocybin, psilocin, baeocystin, or combination thereof in 2.5±1.3 mg/cm².

10. The method of claim 1, wherein the oral dissolvable film has a mass of 175±50 mg.

11. The method of claim 1, wherein the oral dissolvable film has the following dimensions: 44±6 mm×22±3 mm×0.12±0.02 mm.

12. The method of claim 1, wherein the oral dissolvable film has a thickness of less than 0.350 mm.

13. The method of claim 1, wherein the oral dissolvable film comprising the psilocybin, psilocin, baeocystin, or combination thereof in 2.5±1 mg/cm².

14. The method of claim 1, wherein the oral dissolvable film comprises the psilocybin, psilocin, baeocystin, or combination thereof in 2.5±0.5 mg/cm².

15. The method of claim 1, wherein the oral dissolvable film comprises less than 5 wt. % variance of the psilocybin, psilocin, baeocystin, or combination thereof, per unit area of the oral dissolvable film.

16. The method of claim 1, wherein the oral dissolvable film has a content uniformity, such that among two or more samples, the amount of the psilocybin, psilocin, baeocystin, or combination thereof ranges from 85% to 115%, with the standard deviation of less than or equal to 6%.

17. The method of claim 1, wherein the flowable water-soluble or water swellable film-forming matrix that includes a polymer comprises each of plasticizer, binder, preservative, and solvent.

18. The method of claim 1, wherein the flowable water-soluble or water swellable film-forming matrix that includes a polymer further comprises at least one of coloring agent, flavoring agent, sweetening agent, filler, bulking agent, saliva stimulating agent, stabilizing and thickening agent, gelling agent, taste masking agent, pigment, lubricant, release modifier, adjuvant, solubilizer & emulsifier, fragrance, emulsifier, surfactant, pH adjusting agent, buffering agent, lipid, glidant, stabilizer, antioxidant, anti-tacking agent, and humectant.

19. The method of claim 1, wherein the oral dissolvable film comprises:
(a) plasticizer,
(b) solvent,
(c) sweetener,
(d) flavoring agent,
(e) binder,
(f) coloring agent,
(g) preservative, and
(h) psilocybin, psilocin, baeocystin, or combination thereof.

20. The method of claim 1, wherein the oral dissolvable film comprises:
(a) plasticizer selected from the group consisting of glycerol, glycerol monoacetate, diacetate or triacetate, triacetin, polysorbate, cetyl alcohol, propylene glycol, sorbitol, sodium diethylsulfosuccinate, triethyl citrate, and tributyl citrate,
(b) solvent selected from the group consisting of water, ethanol, and combinations thereof,
(c) sweetener,
(d) flavoring agent,
(e) binder selected from the group consisting of pectin, pullulan, starch, pregelatinized starch, gelatin, polyvinylpyrrolidone, methylcellulose, sodium carboxymethylcellulose, ethylcellulose, polyacrylamides, polyvinyloxoazolidone, and polyvinylalcohols,
(f) coloring agent,
(g) preservative selected from the group consisting of benzoate salt, sorbate salt, natamycin, and combinations thereof, and
(h) psilocybin, psilocin, baeocystin, or combination thereof.

21. The method of claim 1, wherein the oral dissolvable film comprises:
(a) 15±10 wt. % plasticizer,
(b) 10±5 wt. % solvent,
(c) 12±8 wt. % sweetener,
(d) 8±7 wt. % flavoring agent,
(e) 30±20 wt. % binder,
(f) 0.02±0.01 wt. % coloring agent,
(g) 0.02±0.01 wt. % preservative, and
(h) 1.25±1 wt. % of psilocybin, psilocin, baeocystin, or combination thereof.

22. The method of claim 1, wherein the oral dissolvable film is configured to dissolve in the oral cavity within 120 seconds.
